(19) United States
(12) Patent Application Publication
(10) Pub. No.: US 2006/0035905 A1
(43) Pub. Date: Feb. 16, 2006

(54) Formulations of phosphodiesterase 5 inhibitors and methods of use

(75) Inventors: Ge Jiang, Thousand Oaks, CA (US); Vincent J. Sullivan, Cary, NC (US); Ronald Pettis, Cary, NC (US); Chorng-Fure Robin Hwang, Thousand Oaks, CA (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 11/042,012

(22) Filed: Jan. 25, 2005

Related U.S. Application Data
(60) Provisional application No. 60/541,916, filed on Feb. 6, 2004.

(51) Int. Cl. A61K 31/519

(52) U.S. Cl. 514/252.16

(57) ABSTRACT

The present invention provides compositions and delivery methods to enhance treatment for sexual dysfunction or hypofunction through the delivery of phosphodiesterase 5 inhibitors to a mammal. Phosphodiesterase 5 inhibitors are one example of a compound class used for this indication. Examples of compounds in this class include taldalafil and vardenafil and sildenafil. Furthermore, the present invention provides compositions and delivery methods to enhance the sildenafil concentration in solution, suspension and gel formulations and methods of parenteral, intradermal, sublingual, intranasal, and buccal sildenafil delivery.

This application claims the benefit of priority of U.S. Provisional Application No. 60/541,916 filed Feb. 6, 2004, which is incorporated herein by reference in its entirety.

### BACKGROUND OF THE INVENTION

1. Field of the Invention

The invention relates to new formulations and delivery routes for phosphodiesterase 5 inhibitors (PDE5), compounds indicated for the treatment of sexual dysfunction or hypofunction, in particular sildenafil.

2. Background Information

In recent years, compositions of sildenafil for oral administration have been widely used for the treatment of erectile dysfunction (ED). However, oral administration of current formulations of sildenafil exhibits low bioavailability and slow uptake. As a result, only about 12% of administered dose enters systemic circulation within one hour after dosing, the period in which the drug effect is highly desired for most patients. Additionally, heavy food uptake further delays the onset of drug action and reduces the bioavailability. Recently, an intranasal spray was reported to enhance the rate of uptake (Hussain et al. Method of Administration of Sildenafil to Produce Instantaneous Response for the Treatment of Erectile Dysfunction, U.S. Pat. No. 6,200,591 B1). Nevertheless, it is possible intranasal delivery may cause potential irritation and irreversible damage to the ciliary action of the nasal cavity from chronic application of nasal dosage forms. The larger intra- and inter-subject variability in mucus secretion in the nasal mucosa, could significantly affect drug absorption from this site. In addition, delivery into the nasal cavity may result in partial oral delivery due to the dose draining into the GI tract. Thus, there remains a need for more improved compositions of sildenafil and more effective methods of administration such as injectable routes and other mucosal routes.

Approaches for delivering substances beneath the surface of the skin have almost exclusively involved transdermal administration, i.e. delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University, New York, 1991). The epidermis is subdivided into five layers or strata of a total thickness of between 75 and 150 μm. Beneath the epidermis lies the dermis, which contains two layers, an outmost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular the papillary dermis, has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue which has been far more easily and predictably targeted than the dermis has been. On the other hand, large molecules such as protein are typically not well absorbed through the capillary epithelium regardless of the degree of vascularity so that one would not have expected to achieve a significant absorption advantage over subcutaneous administration by the more difficult to achieve intradermal administration even for large molecules.

The oral cavity has been highly acceptable to patients as a site for drug delivery. The oral mucosa is relatively permeable with a rich blood supply, it is robust and shows short recovery times after stress or damage, and due to the virtual lack of Langerhans cells, it is tolerant to potential allergens. Furthermore, oral transmucosal drug delivery bypasses first pass effect and avoids pre-systemic elimination in the GI tract. Three different categories of drug delivery fall within the oral cavity: sublingual, buccal, and local, of which the first two sites are feasible for systemic drug delivery.

### SUMMARY OF THE INVENTION

In a general embodiment, the present invention provides compositions and delivery methods to enhance treatment for sexual dysfunction or hypofunction through the delivery of phosphodiesterase 5 inhibitors to a mammal. Phosphodiesterase 5 inhibitors are one example of a compound class used for this indication. Examples of compounds in this class include taldalafil and vardenafil. A particularly preferred example is sildenafil.

The present invention provides compositions and delivery methods to enhance the sildenafil concentration in solution, suspension and gel formulations and methods of parenteral and buccal sildenafil delivery.

In one embodiment, sildenafil citrate is converted into alternative salt forms with enhanced solubility characteristics. In another embodiment, sildenafil was formulated into a flowable and injectable suspension dosage form without the necessity for solubilizing the sildenafil component. Enhanced solubility or enhanced concentration in a flowable format enable a sufficiently high dose to be delivered through intradermal, buccal and intranasal routes. For example, the drug concentration levels achieved by these formulations allow an effective dose to be delivered in a volume suitable for intradermal (ID) delivery through microneedle devices.

The invention also provides a composition comprising a sildenafil acetate, mesylate, or esylate solution, a composition comprising a suspension of sildenafil in Cremorphor, and a composition comprising a suspension of sildenafil in Gelucire.

In further embodiments, the invention provides methods of enhancing efficacy of sildenafil by intradermal, subcutaneous (SC) and buccal delivery. In particular, ID and buccal delivery of sildenafil ensure drug absorption from the skin and buccal membrane, respectively, thus offering fast uptake and high bioavailability which circumvents intranasal and oral delivery problems outlined above. These delivery methods offer a much more rapid onset of pharmacological action than oral administration, while maintaining a minimally invasive character.

The thickness of the dermis and epidermis varies from individual to individual, and within an individual, at different locations on the body. For example, the dermis varies in thickness ranging from just below the epidermis to a depth of from less than 1 mm in some regions of the body to just under 2 to about 4 mm in other regions of the body depending upon the particular study report (Hwang et al., Ann Plastic Surg 46:327-331, 2001; Southwood, Plast. Reconstr. Surg 15:423-429, 1955; Rushmer et al., Science 154:343-348, 1966).

As used herein, intradermal is intended to mean administration of a substance into the dermis in such a manner that the substance readily reaches the richly vascularized dermis and is rapidly absorbed into the blood capillaries and/or lymphatic vessels to become systemically bioavailable. It is believed that placement of a substance predominately at a depth of at least about 0.3 mm, more preferably, at least about 0.4 mm and most preferably at least about 0.5 mm up to a depth of no more than about 3.0 mm, more preferably, no more than about 2.0 mm and most preferably no more than about 1.7 mm will result in rapid absorption of the substance. Placement of the substance predominately at greater depths is believed to result in the substance being more slowly absorbed in the subcutaneous region. The controlled delivery of a substance in this dermal space should enable an efficient migration of the substance to the vascular and lymphatic microcapillary bed, where it can be absorbed into systemic circulation.

Delivery of the substance to deeper tissue depth below the ID space (e.g. SC or IM) may also be beneficial for delivery but may exhibit altered pharmacokinetic (PK) or pharmacodynarnic (PD) effects. As used herein, subcutaneous (SC) is intended to mean the tissue lying below the epidermis and dermis, also referred to as the hypodermis, which is composed of connective tissue and fatty tissue.

The test subject of parenteral delivery of the present invention is a mammal, preferably a human.

As the oral mucosa is highly vascularized and permeable, drug delivery through the membranes lining the sublingual and buccal mucosa may be a desirable route of drug delivery. The concentration enhancement achieved with these sildenafil formulations can also be potentially used in buccal or sublingual delivery dosage forms such as polymeric gels. The polymers can include cationic polymers such as chitosan, neutral materials such as poly(ethylene oxide) (PEO), and anionic polymers such as sodium carboxymethylcellulose and carbopol (I. G. Needleman, et al, Biomaterials 16 (1995) 617-624). The buccal adhesive gel can be squeezed from a tube and applied to the buccal membrane. These gels can also be further processed such as being molded into a certain shape, e.g., a disk, and then lyophilized (M. Artusi et al, Inter. J. Pharm. 250 (2003) 203-213). Such disks can adhere to the buccal membranes, enabling effective buccal delivery.

### BRIEF DESCRIPTION OF THE DRAWINGS

FIG. 1 shows a comparison of sildenafil delivery via IN, ID, SC in rats, along with oral delivery results in human as reported in the Physician's Desk Reference (PDR), Edition 56, 2002, page 2732.

FIG. 2 shows pharmacokinetic profiles of sildenafil solutions via IV, ID or SC routes of administration in Yucatan minipigs (n=6 individuals). A. IV sildenafil mesylate solution; B. ID 1.5 mm needle length, sildenafil mesylate solution; C. ID 1.5 mm needle length, sildenafil acetate solution; D. ID 2.0 mm needle length, sildenafil mesylate solution; E. SC sildenafil mesylate solution; F. Comparison of different routes and salt forms (Average of 6 minipigs).

FIG. 3 displays pharmacokinetic profiles of sildenafil suspensions via ID and SC delivery in Yucatan minipigs (n=6 individuals). A. ID 1.5 mm sildenafil suspension in Cremorphor EL; B. SC sildenafil suspension in Cremorphor EL; C. ID 1.5 mm sildenafil suspension in Gelucire 50/13; D. SC sildenafil suspension in Gelucire 50/13; E. Comparison of different suspensions and routes (Average of 6 minipigs).

FIG. 4 is a partial cross-sectional illustration of a needle assembly designed for ID delivery of compounds according to aspects of the invention.

FIG. 5 A perspective view of one technique for making an ID injection according to one aspect of the invention.

### DETAILED DESCRIPTION OF THE INVENTION

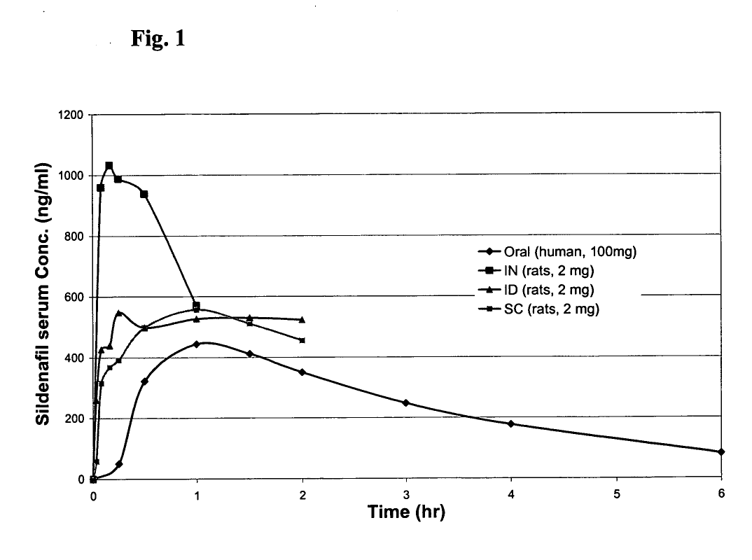

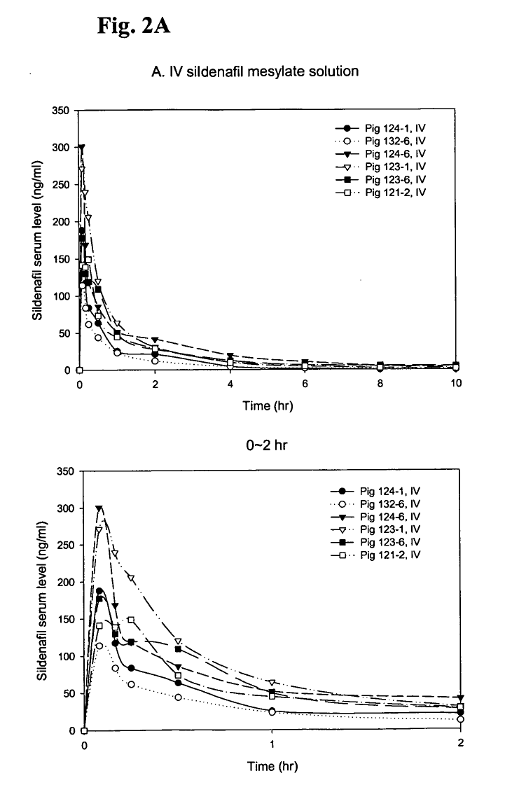

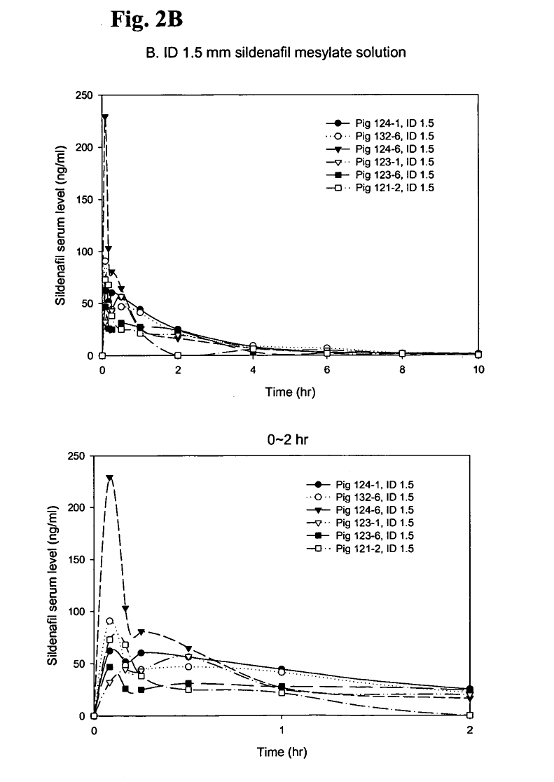

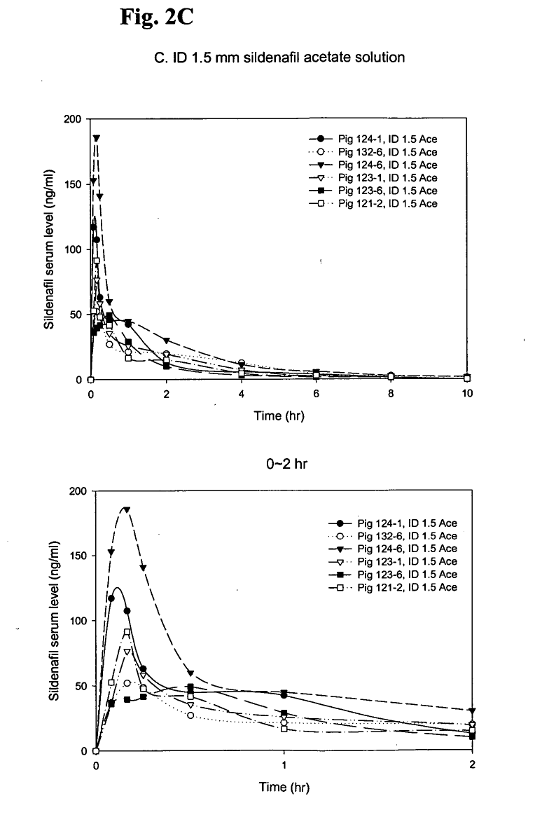

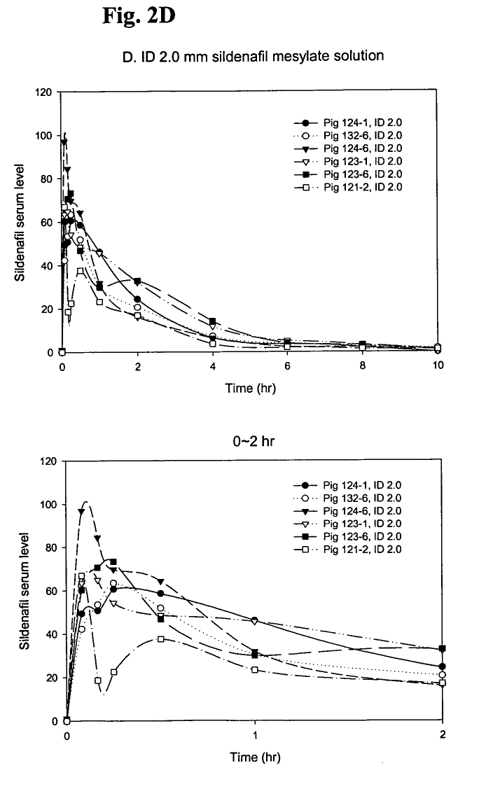

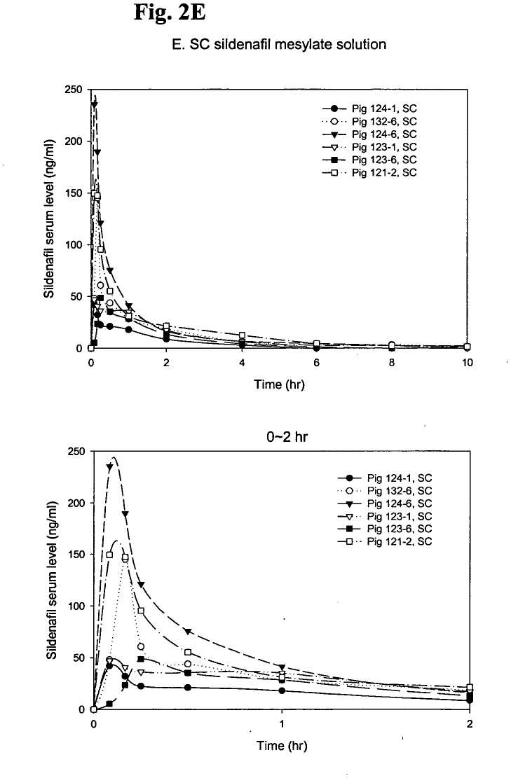

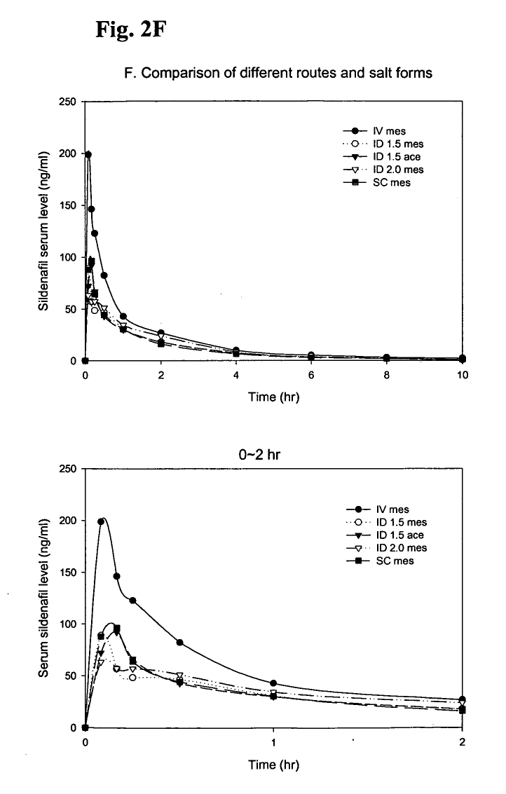

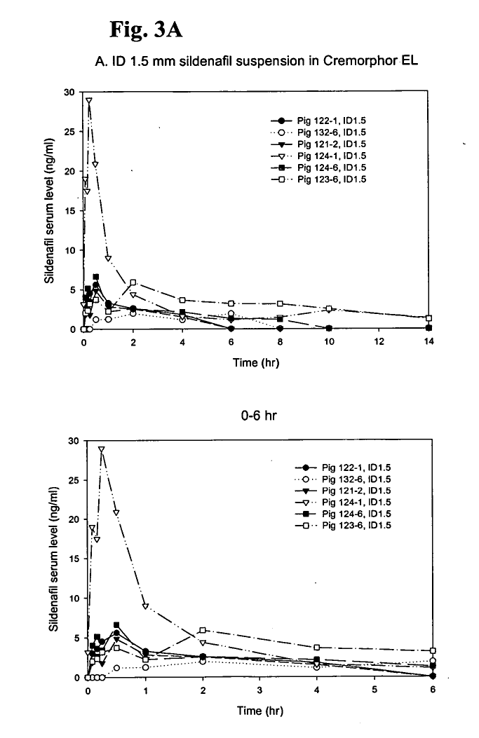

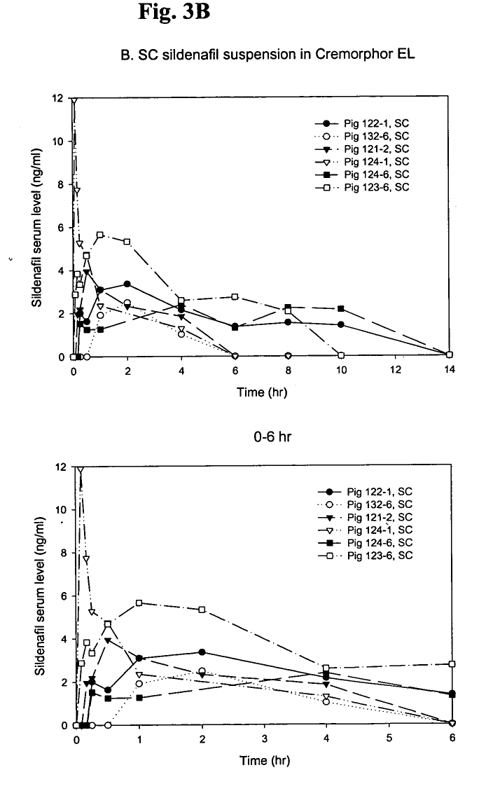

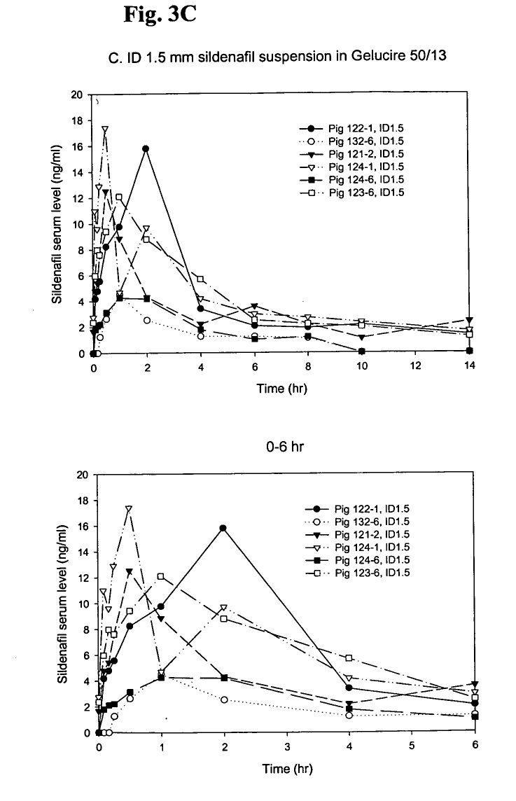

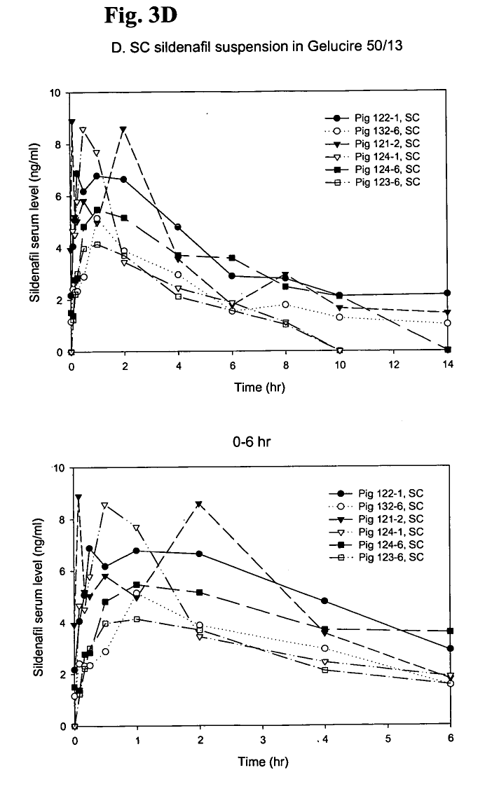

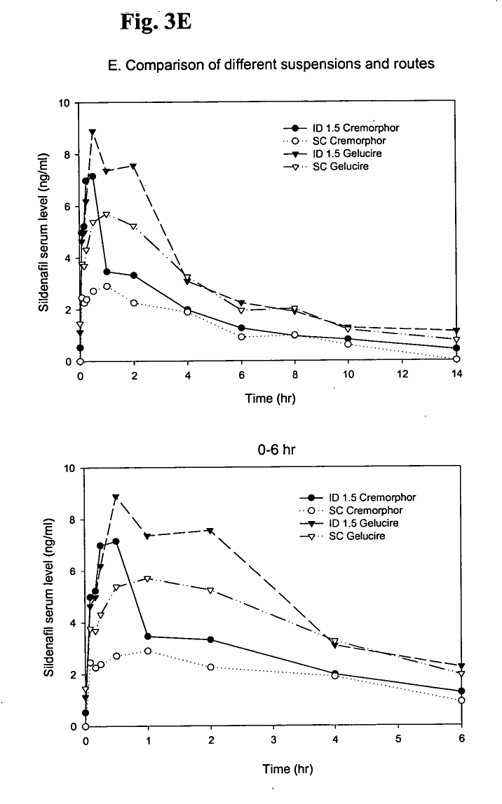

Studies were carried out in Sprague-Dawley rats (FIG. 1) and in Yorkshire and Yucatan pigs. Pharmacokinetic studies (FIGS. 2 and 3, Table 1 and 2) were conducted in Yucatan pigs. Tissue irritancy study testing (Table 3) was conducted in Yorkshire pigs.

### EXAMPLE 1

Comparison of Routes of Administration of Sildenafil in Sprague-Dawley Rats

IN, ID, SC and IV pharmacokinetic (PK) studies were performed in Sprague-Dawley rats.

Formulation

Preparation of sildenafil base from sildenafil citrate: Viagra® tablets were ground into powder and 5% methanesulfonic acid (CH₃SO₃H) was added dropwise to the powder to achieve a pH of 1˜2 in the resulting slurry. The slurry was filtered and 3% NH₄OH was added dropwise to the filtrate to reach pH 8˜9, resulting in precipitation of sildenafil base. The precipitated base was collected by filtering and vacuum drying.

Preparation of sildenafil mesylate solution: 60 mg of sildenafil base was dissolved in 540 μl 2% CH₃SO₃H, then 1860 μl pH 4.0 0.1M acetate buffer was added. The final concentration of sildenafil was 25 mg/ml with a pH 3.5. This method enabled an approximately 10 fold increase in solubility compared to sildenafil citrate.

Rat Animal Model

Animal surgery: The rat's femoral vein was cannulated for blood sampling for every group. For IN rats, an incision was made in the trachea and a polypropylene tube was inserted as an airway. To seal the pass way between the nasal cavity and trachea, a sealed tube was inserted through the same incision into the posterior of nasal cavity. Finally the nasopalatine was closed with an adhesive.

Drug administration: 80 μl of sildenafil mesylate solution was administered to rats via IN, ID and SC by a blunt needle, ID 1 mm 34 G needle and regular 31 G needle, respectively.

Blood samples were drawn from the femoral vein at predetermined time points. Serum samples were collected after centrifugation and stored at −70° C. until analysis.

Analysis of Sildenafil Serum Level in Rats

20 μl of 12% perchloric acid was added to 80 μl rat serum followed by vortexing. Then 100 μl of water was added to achieve the sample volume required by HPLC assay. The above mixture was centrifuged and the supernatant was analyzed by HPLC at the following condition:
Mobile phase—Acetonitrile/0.067M K₂PO₄ buffer (pH 2.6 adjusted by H₃PO₄) 71.5/28.5
Column—Phenomenex luna® 5μ, C18 (2), 250×4.6
Flow rate—1.0 ml/min
Detection—UV 230 nm.
   Sildenafil PK Results in a Rat Model

Following IN administration, sildenafil was rapidly absorbed into systemic circulation, and the peak serum concentration occurred at approximately 10 minutes with Cₘₐₓ approximately 1000 ng/ml, roughly twice that of ID and SC. For ID administration, sildenafil level of 428 ng/ml was reached at 5 min and the maximum systemic concentration was reached around 15 min with a Cₘₐₓ of 547 ng/ml. From 30 min to 120 min, sildenafil serum levels remained fairly constant around 520 ng/ml, suggesting the absorption and elimination occurred simultaneously and almost at the same rate. SC absorption was slower than ID, the Cₘₐₓ appearing at about 30˜60 min with a comparable maximum serum drug level to the ID group. All three routes, IN, ID and SC in rats, demonstrated faster drug absorption in comparison to the previously reported human clinical results for oral delivery.

### EXAMPLE 2

PK Study of Sildenafil Solutions in Yucatan Pigs

A pharmacokinetic (PK) study with solution formulations was carried out in Yucatan minipigs (n=6) and serum samples were analyzed for sildenafil content using a HPLC/MS/MS method.

Formulation

Sildenafil mesylate solution: 50 mg sildenafil base prepared as described in Example 1 was dissolved in 450 μl 1.6% CH₃SO₃H, then mixed with 1550 μl 0.1M acetate buffer (pH 4.0), finally 11.0 mg NaCl was added to adjust the tonicity to ˜290 mOsm/kg.

Sildenafil acetate solution: 20 mg sildenafil base dissolved in 200 μl 5% CH₃COOH, then mixed with 600 μl 0.1M acetate buffer (pH 4.0), finally 3.7 mg NaCl was added to adjust the tonicity to ˜290 mOsm/kg.

Minipig PK Model

Intradermal injections were conducted at the flank region of Yucatan pigs using two different length of 31 gauge microneedles, 1.5 mm and 2.0 mm, noted as ID1.5 and ED 2.0, respectively. SC injections were conducted using regular half inch 30 gauge needles also at the flank region. The injection volume was 200 μl (containing 5 mg sildenafil) for each pig. After drug administration, blood samples were taken from the jugular vein port periodically and sera were collected after centrifugation and stored at −70° C. until analysis.

Analysis of Sildenafil Serum Level in Minipigs

Pig serum samples (including pooled untreated serum) were briefly vortex-mixed and a 100 μl volume was transferred into a borosilicate glass screw-top conical test tube. 10 μl of the appropriate spiking solution was added to calibration and quality control (QC) samples. 10 μl of 50:50 methanol-water was added to study samples. Then 10 μl of a sildenafil derived internal standard solution (200 μg/ml) was added and voltex-mixed for 30 Sec. The subsequent liquid/liquid extraction was performed by adding 2 ml of methyl t-butyl ether (MTBE) to each tube and vortex-mixing for 1 min, followed centrifugation at 3000 rpm for ˜10 min. Then the aqueous layer was frozen in dry-ice acetone bath and MTBE layer was decanted to glass tubes and dried in the TurboVap at 35° C. The samples were reconstituted in 200 μl acetonitrile containing 1% acetic acid and 0.025% TFA for LC-MS-MS analysis.

The standards and QCs for LC-MS-MS were made from a stock solution in MeOH-water 1:1, v/v. Calibration standard at concentrations of 10, 20, 500, 200, 500, 1000, 2500 and 5000 ng/ml. QCs were prepared at levels of 20, 2000, 4000 ng/ml. All standard and QCs were aliquoted and stored at 2-8° C. The LC-MS-MS system consisted of a Shimazu HPLC system (Kyoto, Japan) and a Sciex API 3000 tandem mass spectrometer (Ontario, Canada) with electrospray ionization in the positive ion mode [(+)-ESI]. The analytical column, Betasil silica of 5 μm, 50×3 mm I.D., was purchased from Keystone Scientific (Bellefonte, Pa., USA) and operated at 30° C. The mobile phase was a mixture of acetonitrile/water at 15/85 v/v and contains 1% acetic acid and 0.025% TFA. The injection volume was 30 μl. The run time was 3 min and the flow was 0.3 ml/min. The mass spectrometer was operated under multiple reaction monitoring mode with the ionspray needle maintained at 1.5 kV. The turbo gas temperature was 400° C. Nebulizing gas, curtain gas, and collision gas flows were at instrument settings of 8, 8, and 12, respectively. The declustering potential (DP), focusing potential (DP) and collision energy (CE) were at 65, 240, and 54 V, respectively. The transitions (precursor to product) monitored were m/z 475.1 to 297.2 for sildenafil.

PK Results of Sildenafil Solutions in Yucatan Minipigs

The results were summarized and plotted in FIG. 2. FIG. 2 a, b, c, d, e represent IV mesylate, ID 1.5 mesylate, ID 1.5 acetate, ID 2.0 mesylate and SC mesylate, respectively. Even though the six animals exhibited considerable inter-subject variance, the individual swine showed consistent absorption behavior for all routes of administration. For example, pig 124-6 had the highest Cₘₐₓ and AUC of all pigs by all routes of administration. FIG. 2f shows the average of six pigs using different routes and solution forms. The sildenafil serum data was analyzed using a non-compartment model in WinNonlin 4.1 software from Pharsight Corporation and the pharmacokinetic parameters were summarized Table 1.

TABLE 1
PK summary. All data was presented as Average (Standard Error). All
pigs received ˜5 mg of sildenafil regardless of body weight. Area under the curve (AUC),
mean resident time (MRT) were calculated up to the last sampling time point. Clearance
was dose normalized and used for the calculation of bioavalibility.

| | Cₘₐₓ | Tₘₐₓ | AUC | MRT | Terminal | Clearance | Bioavailability^{a} |
|---|---|---|---|---|---|---|---|
| Delivery | (ng/ml) | (hr) | (hr*ng/ml) | (hr) | t_{½}(hr) | (ml/hr/kg) | (%) |
| IV | 307.35 | 0.0^{b} | 229.41 | 1.90 | 3.01 | 1413.48 | NA |
| mesylate^{a} | (63.87) | (0.0) | (29.86) | (0.30) | (0.71) | (204.06) | |
| ID 1.5 mm | 93.13 | 0.083^{c} | 116.21 | 1.90 | 2.27 | 2537.01 | 56.95^{c} |
| Mesylate | (27.93) | (0.0) | (9.73) | (0.09) | (0.28) | (213.70) | (12.08) |
| ID 2.0 mm | 70.98 | 0.18 | 133.56 | 2.27 | 3.83 | 2143.21 | 66.79^{c} |
| mesylate | (5.47) | (0.03) | (12.92) | (0.16) | (0.54) | (172.13) | (8.02) |
| SC mesylate | 111.28 | 0.12 | 120.13 | 2.43 | 2.84 | 2740.31 | 54.77^{c} |
| | (32.06) | (0.03) | (21.54) | (0.33) | (0.52) | (522.22) | (7.76) |
| ID 1.5 mm | 95.35 | 0.21 | 114.97 | 1.85 | 2.19 | 2496.84 | 59.21^{c} |
| acetate | (20.87) | (0.06) | (16.91) | (0.16) | (0.17) | (281.37) | (10.30) |

^{a}Pig # 132-6 was excluded from all IV mesylate values due to a lower IV AUC compared to other routes
^{b}Pig # 121-2 was regarded as an outlier and excluded. Its Tmax (0.25 hr) was higher than mean ± 2SD of others.
^{c}Pig # 123-1 was regarded as a out layer and excluded. Its Tmax (0.5 hr) was higher than mean ± 2SD of others.

The results shown in Table 1 and FIG. 2 support the following conclusions: The PK profiles of mesylate salt by ID 1.5 mm and SC administrations were similar (no significant statistical significance at the 95% confidence level), though ID appears to have a slightly shorter tₘₐₓ. Rapid absorption occurred by both ID and SC (ca. 5 min and 7 min to Cₘₐₓ for ID and SC, respectively) and bioavailability was ˜55% in both cases. ID delivery at 2.0 mm shows a bi-phasic pattern, with a similar tₘₐₓ at the initial peak but slower drop off in drug level compared to ID 1.5 mm. This pattern could be useful for the need to prolong the drug effect in some patients. ID 2.0 mm also resulted in less erythema and edema (data not shown) in comparison to ID 1.5 mm. For ID 1.5 mm injections, onset of mesylate salt was slightly more rapid than acetate salt (i.e., tₘₐₓ of 5 min and 12.6 min for mesylate and acetate, respectively.) The tissue irritancy in Yucatan pigs with mesylate salt was significantly less than acetate salt. For instance, at 24 hr after injection, only negligible or slight level of erythema and edema were observed with mesylate versus tissue necrosis and scaring observed in some pigs receiving acetate salt. The bioavailability was comparable (54˜67%) for ID or SC for both mesylate or acetate salt forms.

### EXAMPLE 3

Study of PK Profiles Sildenafil Suspensions in Yucatan Minipigs

Sildenafil suspensions in Cremorphor EL and in Gelucire 50/13 were prepared to determine whether suspensions could prolong the PK profile.

Formulation

Preparation of sildenafil suspension in Cremorphor EL: Add 0.8 ml of Cremorphor EL to 80 mg sildenafil base, heat the mixture to 75° C. and keep at 75° C. for 3 hrs. Vortex a few times during the heating. Meanwhile, heat saline to 75° C. Add 2.4 ml saline to the sildenafil/Cremorphor mixture. Vortex, cool to room temperature. Vortex before injection into swine.

Preparation of sildenafil suspension in Gelucire 50/13: Heat Gelucire 50/13 to 75° C. to melt. Add 0.8 ml of melted Gelucire to 80 mg sildenafil base, heat the mixture to 75° C. and keep at 75° C. for 3 hrs. Vortex a few times during the heating. Meanwhile, heat saline to 75° C. Add 2.4 ml saline to the sildenafil/Gelucire mixture. Vortex, cool to room temperature. The suspension can be warmed to 37° C. in order to allow it to be drawn into a syringe before injection into swine.

Minipig PK Model

Intradermal injections were conducted at the flank region of Yucatan pigs using 30 gauge 1.5 mm BD microneedles. SC injections were conducted using regular half inch gauge needles also at the flank region. The injection volume was 200 μl (containing 5 mg sildenafil) for each pig. After drug administration, blood samples were taken from the jugular vein port periodically and sera were collected after centrifugation and stored at −70° C. until analysis.

Analysis of Sildenafil Serum Level in Minipigs

Same as that in Example 2.

PK Results of Sildenafil Suspensions in Yucatan Minipigs

The results were summarized and plotted in FIG. 3. FIG. 3 a, b, c, d, e representing ID 1.5 Cremorphor, S C Cremorphor, ID 1.5 Gelucire, S C Gelucire, respectively. FIG. 3f plotted the average of six pigs using different routes and solution forms. The sildenafil serum data was analyzed using a non-compartment model in WinNonlin 4.1 and the pharmacokinetic parameters were summarized Table 2.

TABLE 2
PK summary of sildenafil suspensions. IV data of sildenafil mesylate
solution in Table 1 was used as the control to calculate bioavailability. All data was
presented as Average (Standard Error). All pigs received ˜5 mg of sildenafil regardless of
body weight. AUC, AUMC, MRT were calculated up to the last sampling time point.
Clearance was dose normalized and used for the calculation of bioavailability.

| | Cₘₐₓ | Tₘₐₓ | AUC | MRT | Terminal | Clearance | Bioavailability |
|---|---|---|---|---|---|---|---|
| Delivery | (ng/ml) | (hr) | (hr*ng/ml) | (hr) | t_{½}(hr) | (ml/hr/kg) | (%)^{a} |
| IV | 307.35 | 0.0^{b} | 229.41 | 1.90 | 3.01 | 1413.48 | NA |
| mesylate^{a} | (63.87) | (0.0) | (29.86) | (0.30) | (0.71) | (204.06) | |
| ID 1.5 mm | 8.99 | 0.96 | 27.46 | 4.35 | 3.32 | 8476.80 | 30.21 |
| Cremorphor | (4.05) | (0.33) | (10.00) | (1.26) | (0.35) | (1703.09) | (10.11) |
| SC | 4.96 | 1.60 | 15.37 | 3.04 | 2.66 | 12629.12 | 13.72 |
| Cremorphor | (1.47) | (0.58) | (3.30) | (0.66) | (0.37) | (2880.92) | (1.45) |
| ID 1.5 mm | 11.09 | 1.00 | 49.81 | 5.52 | 3.47 | 5661.09 | 39.75 |
| Gelucire | (2.27) | (0.22) | (11.60) | (0.88) | (0.54) | (1851.78) | (11.16) |
| SC | 6.52 | 0.64 | 40.69 | 5.42 | 4.89 | 4983.39 | 33.37 |
| Gelucire | (0.79) | (0.17) | (8.80) | (1.16) | (0.48) | (985.50) | (5.23) |

^{a}Pig # 132-6 was excluded from all IV mesylate values due to a lower IV AUC compared to other routes
^{b}Pig # 121-2 was regarded as an outlier and excluded. Its Tmax (0.25 hr) was higher than mean ± 2SD of others.

Results reported in Table 2 and FIG. 3f support the following conclusions: The suspension forms of sildenafil significantly extended the absorption time in comparison to the solution forms in FIG. 2. This extension is reflected by the longer tₘₐₓ and longer mean resident time (MRT) than previously observed tₘₐₓ and MRT with solution formulations used in Example 2. The absorption of suspensions was more gradual than solutions, resulting in a lower Cₘₐₓ and a more prolonged PK profile. Therefore, suspension formulations are good candidates when a sustained treatment is desired. For both suspensions, ID delivery showed a higher bioavailability than the same formulation delivered by SC route. This effect is especially pronounced with the Cremorphor suspension where the average bioavailability for ID is more than two folds of that of SC (30.21% versus 13.72%). Overall the suspensions gave rise to a lower bioavailability than the solution forms. The bioavailabiltiy observed for suspensions were ≦40%, versus≧55% for solutions. The Gelucire suspension gave rise to a higher bioavailability that Cremorphor regardless of injection route.

### EXAMPLE 4

Study of Local Irritancy of Sildenafil Solutions and Suspensions in Yorkshire Swine.

The formulations of sildenafil suspension and solution of different salt forms were also tested for injection site irritancy in another swine species, Yorkshire swine. The results are shown in Table 3.

Formulation

D (mesylate solution): 10 mg sildenafil base dissolved in 90 μl 2% CH₃SO₃H, then mixed with 310 μl 0.1M acetate buffer (pH 4.0) [Vehicle control: 90 μl 2% CH₃SO₃H mixed with 310 μl 0.1M acetate buffer (pH 4.0)]
K (esylate solution 1): 10 mg sildenafil base dissolved in 90 μl 2% C₂H₅SO₃H, then mixed with 310 μl 0.1M citrate phosphate buffer (pH 4.0) [Vehicle control: 90 μl 2% C₂H₅SO₃H mixed with 310 μl 0.1M citrate phosphate buffer (pH 4.0)]
M (esylate solution 2): 10 mg sildenafil base dissolved in 90 μl 2% C₂H₅SO₃H, then mixed with 310 μl 0.1M glycine-HCl (pH 3.6) [Vehicle control: 90 μl 2% C₂H₅SO₃H mixed with 310 μl 0.1M glycine-HCl buffer (pH 3.6)]
O (Cremorphor suspension 1). Add sildenafil base 10 mg to Cremorphor EL 100 μl, then heat the mixture to 75° C., maintain at 75° C. for 3 hrs, vortex 3 times in between, then mixed with saline 300 μl (also heated to 75° C.). [Vehicle control: Cremorphor EL 100 μl (heated to 75° C.), mixed with saline 300 μl (also heated to 75° C.).]
Q (Cremorphor suspension 2). Add sildenafil base 10 mg to Cremorphor EL 100 μl, then heat the mixture to 75° C., maintain at 75° C. for 3 hrs, vortex 3 times in between, then mixed with citrate phosphate buffer (pH 4.0) 300 μl (also heated to 75° C.). [Vehicle control: Cremorphor EL 100 μl (heated to 75° C.), mixed with citrate phosphate buffer (pH 4.0) 300 μl (also heated to 75° C.).]
S (Cremorphor suspension 3). Add sildenafil base 10 mg to Cremorphor EL 100 μl, then heat the mixture to 75° C., maintain at 75° C. for 3 hrs, vortex 3 times in between, then mixed with glycine-HCl buffer 300 μl (also heated to 75° C.). [Vehicle control: Cremorphor EL 100 μl (heated to 75° C.), mixed with glycine-HCl buffer (pH 3.6) 300 μl (also heated to 75° C.).]
   Yorkshire Swine Dermal Irritancy Model

Each of the above formulations were injected in the flank region of a Yorkshire pig by Mantoux technique using a 25 G needle. The injection volume was 100 μl. Table 3 shows tissue irritancy as measured by Draize scoring in Yorkshire pigs of several formulations of sildenafil mesylate and, esylate solutions, and sildenafil suspensions in cremorphor.

Yorkshire Swine Dermal Irritancy Results

The results show that the solution form of different salts elicited similar levels of irritancy and other dermal effects. The Cremorphor suspensions of sildenafil only resulted in erythema, but not tissue necrosis, showing a significantly lower degree of tissue irritancy than the solution forms. Similar levels of tissue reaction were elicited by sildenafil cremorphor suspension in the various aqueous vehicles, i.e., saline, citrate phosphate buffer, or glycine-HCl buffer.

TABLE 3
Irritation at injection site of sildenafil solution and suspension formulations

| Sildenafil | Draize Score^{a} of | Draize Score^{a} of | Visible necrosis^{b} | Visible necrosis^{b} |
|---|---|---|---|---|
| Formulation | Formulation | vehicle control | of Formulation | of vehicle control |
| D | 2.0 | 1.0 | ++ | + |
| K | 2.0 | 1.0 | ++ | + |
| M | 2.0 | 1.0 | ++ | + |
| 0 | 2.0 | 2.0 | − | − |
| Q | 2.0 | 2.0 | − | − |
| S | 2.0 | 2.0 | − | − |

^{a}The first number in draize score indicates erythema at 0-4 scale. The second number in draize score indicates edema at 0-4 scale.
^{b}The extent of necrosis was indicated by the number of “+”. “−” means no necrosis observed.

The compositions of certain aspects of the invention may be administered using any of the devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

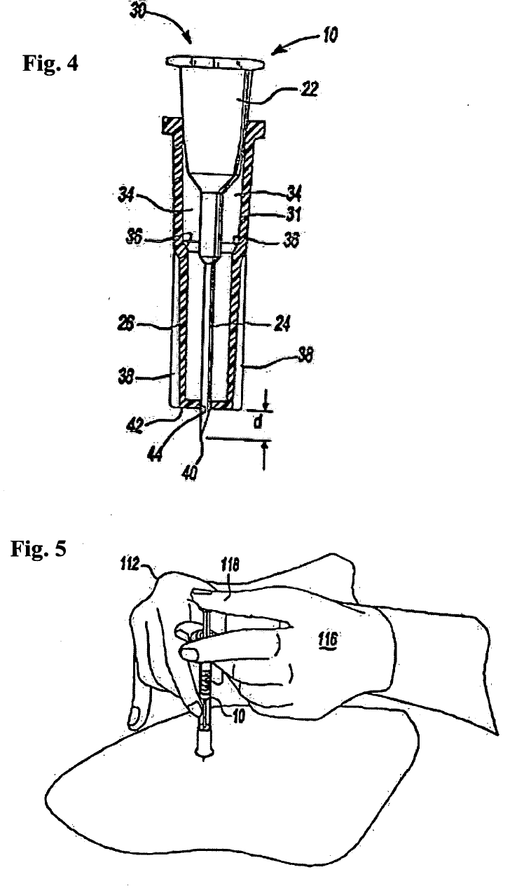

In a specific embodiment, the invention encompasses a drug delivery device as disclosed in FIG. 4 which illustrates an example of a drug delivery device which can be used to practice the methods of certain aspects of the present invention for making intradermal injections, as further illustrated in FIG. 5. The device illustrated in FIG. 4. includes a needle assembly 10 which can be attached to a syringe barrel. Other forms of delivery devices may be used including pens of the types disclosed in U.S. Pat. No. 5,279,586, U.S. patent application Ser. No. 09/027,607 and PCT Application No. WO 00/09135, the disclosures of which are hereby incorporated by reference in their entirety.

The needle assembly 10 includes a hub 22 that supports a needle cannula 24. The limiter 26 receives at least a portion of the hub 22 so that the limiter 26 generally surrounds the needle cannula 24. One end 30 of the hub 22 is able to be secured to a receiver 32 of a syringe or other fluid supply device. A variety of syringe types for containing the substance to be intradermally delivered according to aspects of the present invention can be used with a needle assembly designed, with several examples being given below. The opposite end of the hub 22 optionally includes extensions 34 that are nestingly received against abutment surfaces 36 within the limiter 26. A plurality of ribs 38 optionally are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal, and the outlet depth is controlled. The limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIG. 4 illustrates a two-piece assembly where the hub 22 is made separate from the limiter 26, devices for use in connection with certain aspects of the invention are not limited to such an arrangement. Forming the hub 22 and limiter 26 integrally from a single piece of material is an alternative to the example shown in FIG. 4. Additionally, it is possible to adhesively or otherwise secure the hub 22 to the limiter 26 in the position illustrated in FIG. 4 so that the needle assembly 10 becomes a single piece unit upon assembly.

As shown in FIG. 5 the needle 10 and syringe may be grasped with a first hand 112 and the plunger of the syringe depressed with the thumb 118 of a second hand 116.

It will be apparent that the present invention has been described herein with reference to certain preferred or exemplary embodiments. The preferred or exemplary embodiments described herein may be modified, changed, added to, or deviated from without departing from the intent, spirit and scope of the present invention.

1. A composition comprising an aqueous solution of a phosphodiesterase 5 inhibitor in the form of a salt, at a concentration of 1 to 200 mg/ml, and a pH between about 2.5 and about 5, wherein the salt is either an acetate or ethylate salt.

2. The composition of claim 1 wherein the phosphodiesterase 5 inhibitor is sildenafil.

3. A composition comprising a suspension of a phosphodiesterase 5 inhibitor particles at a concentration of 1 to 400 mg/ml in an aqueous suspension containing a pharmaceutically acceptable vehicle.

4. The composition of claim 3 wherein the phosphodiesterase 5 inhibitor is sildenafil.

5. The composition of claim 3 wherein the suspension contains ethoxylated castor oil (e.g. Cremophor EL or ELP) at a concentration of 5 to 99 weight %.

6. A composition comprising a phosphodiesterase 5 inhibitor, at a concentration of 1 to 400 mg/ml in a gel or viscous matrix.

7. The composition of claim 6 wherein said phosphodiesterase 5 inhibitor is sildenafil.

8. The composition of claim 6 wherein the gel or viscous matrix contains stearoyl macrogolglycerides (e.g. Gelucire 50/13) at a concentration of 5 to 99 weight %.

9. A method of administering a phosphodiesterase 5 inhibitor comprising parenterally administering a solution, suspension or emulsion composition comprising said phosphodiesterase 5 inhibitor.

10. The method of claim 9 wherein said phosphodiesterase 5 inhibitor is sildenafil.

11. The method of claim 9 wherein said composition is a solution.

12. The method of claim 9 wherein said composition is a suspension.

13. The method of claim 9 wherein said composition is an emulsion.

14. The method of claim 11 wherein the pH of said solution is between about 2.5 and 5.

15. The method of claim 9 wherein said compositions additionally comprise mesylate, acetate or ethylate.

16. The method of claim 9 wherein the composition is administered to skin at a depth between 0.3 and 3.0 mm using a microcannula or microneedle of a gauge between 30 and 34.

17. The method of claim 9 wherein the volume administered is less than 600 microliters.

18. A method of treating erectile dysfunction comprising intradermally administering a pharmaceutically acceptable composition comprising of sildenafil to a subject.

19. A method of administering sildenafil comprising parenterally administering a liquid composition of claim 2.

20. A method of administering sildenafil comprising parenterally administering a liquid composition of claim 4.

21. A method of administering sildenafil comprising parenterally administering a liquid composition of claim 7.

22. A method of administering sildenafil comprising buccally or sublingually administering the liquid composition of claim 2.

23. A method of administering sildenafil comprising buccally or sublingually administering the liquid composition of claim 4.

24. A method of administering sildenafil comprising buccally or sublingually administering the liquid composition of claim 7.

25. A method of treating erectile dysfunction comprising subcutaneously administering a pharmaceutically acceptable composition comprising sildenafil to a subject.
